(19) United States
(12) Patent Application Publication
(10) Pub. No.: US 2015/0037168 A1
(43) Pub. Date: Feb. 5, 2015

(54) UNIVERSAL INFUSION DEVICE FOR LIQUID MEDICINES AND THE LIKE, AND METHOD FOR CONTROLLING THE EROGATION OF SUCH LIQUID MEDICINE AND THE LIKE

(71) Applicant: TECRES S.p.A., Sommacampagna (Verona) (IT)

(72) Inventors: Giovanni Faccioli, Monzambano (MN) (IT); Renzo Soffiatti, Nogara (VR) (IT); Mario Socca, Sommacampagna (Verona) (IT)

(73) Assignee: TECRES S.p.A, Sommacampagna (Verona) (IT)

(21) Appl. No.: 14/382,284

(22) PCT Filed: Dec. 10, 2012
(86) PCT No.: PCT/IB2012/057147

§ 371 Date: Aug. 29, 2014

(30) Foreign Application Priority Data
Mar. 2, 2012 (IT) VR2012A000034

(51) Int. Cl. A61M 5/142; A61M 5/38; F04B 11/00; F04B 43/09; F04B 49/06; F04B 49/20; A61M 5/36; F04B 43/12

(52) U.S. Cl. CPC A61M 5/14232; A61M 5/365; A61M 5/38; F04B 43/12; F04B 43/09; F04B 49/065; F04B 49/20; F04B 11/00; A61M 2205/3331; A61M 2205/0216; A61M 2205/581
USPC 417/18; 417/476; 417/44.2; 417/63; 417/1; 417/53; 417/44.11

(57) ABSTRACT

The present invention concerns an infusion device for liquid medicines and the like, including at least one main body associated with at least one tank of liquid medicine, at least one peristaltic pump having at least one rotor coupled with at least one press roller that can be pushed so as to engage with a tubular duct wound so as to form at least one complete ring, abutting on the inner wall of at least one casing of said peristaltic pump, and including two substantially adjacent portions of said tubular duct. The device includes one control processor, associated with the motor of the rotor, including means for adjusting the speed of the rotor suitable for increasing the speed of said rotor in at least one sector of the complete revolution carried out by said rotor.

### TECHNICAL FIELD OF THE INVENTION

The present invention concerns a universal infuser device for liquid medicines and the like, and a method for controlling the dispensing of liquid medicine and the like.

In particular the present invention concerns an infuser device for liquid medicines, such as drugs, anaesthetics, chemotherapeutics, etc.

### STATE OF THE ART

In the medical field it is known to use devices for infusing liquid medicine such as for example, drugs, anaesthetics, chemotherapeutics and the like, comprising pumping means that dispense a certain flow-rate of liquid along a duct, usually communicating with an infusion needle.

In particular infuser devices are known on the market the pumping means of which comprise a peristaltic pump, actuated by a motor, which makes it possible to obtain a more precise infusion of the liquid medicine with respect to devices with other types of pumping means. Moreover in the peristaltic pump the liquid medicine transiting in the duct is not placed in contact with any other component of the pump itself, so as to protect it from any contamination, which is an absolutely essential aspect in the medical field.

More in detail, peristaltic pumps conventionally used in infuser devices of the known type comprise a tubular duct made from elastically deformable material which is pressed against the inner surface of the body of the pump by press rollers (for example two or even three rollers) associated with a rotor actuated by the motor: the compression of the tubular duct by the press rollers and the subsequent release determine the suction of the liquid medicine from a tank, and the movement of the liquid itself exiting towards the patient.

As mentioned the tubular duct is made from an elastically deformable material, like for example silicone, PVC, or other polymeric materials having suitable mechanical characteristics, which makes it possible for the portion of tubular duct, each time squashed by the passing roller, to quickly return to the initial size.

However, in infuser devices of the known type, the peristaltic pumps used have the drawback of having pulsed dispensing of the liquid medicine at the outlet, due to the fact that the tubular duct is indeed compressed and released many times, each time the rotor carries out a turn, by the passing of the press rollers (as mentioned there can be for example two or even three rollers).

In order to oppose such a phenomenon and to try to obtain a more continuous dispensing of the liquid medicine at the outlet of the pump, a greater number of press rollers can be used: however, a high number of rollers (and the consequent fact of them being so close to one another) would not give enough time for the portion of tubular duct that has just been compressed to return to the initial dimensions, so as to be able to draw the liquid medicine, before the passing of the following roller, and such a solution is thus only effective for low flow-rates of liquid.

In known infuser devices having conventional peristaltic pumps a typical drawback is thus the ratio between minimum and maximum dispensing: indeed, a peristaltic pump that is sized so as to dispense very low quantities of liquid medicine is not able to dispense very large flow-rates, which in some applications are just as necessary as the very low ones.

### SUMMARY OF THE INVENTION

The technical task of the present invention is therefore that of devising an infuser device for liquid medicine and the like and a method for controlling the dispensing of liquid medicine and the like, which allow the dispensing of the liquid medicine in outlet to be continuous and to have any flow-rate required according to the necessary dosage.

In such a technical task, one particular purpose of the present invention is to make an infuser device that is simple and fast to use.

This task and this purpose are achieved by the infuser device for liquid medicine and the like according to thepresent principles.

The infuser device for liquid medicine and the like is of the type comprising at least one main body that is associated with at least one tank of liquid medicine, and comprises at least one peristaltic pump having at least one rotor that is coupled with at least one press roller and that is associated with at least one tubular duct made from elastically deformable material that is placed in communication on one side with the tank and on the other with at least one mouth for dispensing the liquid of the infuser device; the mentioned tubular duct is wound so as to form at least one complete ring abutting on the inner wall of at least one casing of the peristaltic pump and the roller is selectively pushed to engage with at least the complete ring, so as to allow a continuous dispensing of liquid medicine for any flow-rate required.

Moreover, the same purpose is achieved by the method for controlling the dispensing of liquid medicine and the like, according to the present principles.

The method for controlling the dispensing of liquid medicine and the like actuated with the infuser device, comprises the steps of detecting at least the passing of the press roller at the adjacent portions of the tubular duct, dividing the complete revolution carried out by the rotor into a predetermined number of angular sectors, increasing the rotation speed of the rotor, through the control processor, at least in the sector following the detection point of the passing of the roller at the adjacent portions, defining the fastest compression by the roller itself of the corresponding portion of the ring so as to allow continuous dispensing over time of the liquid medicine at the desired flow-rate.

Further advantageous characteristics are described in the dependent claims.

### BRIEF DESCRIPTION OF THE DRAWINGS

The characteristics of the invention shall become clearer by a man skilled in the art from the following description and the attached drawing tables, given as an example and not for limiting purposes, in which:

FIG. 1 is a perspective view of the infuser device for liquid medicine and the like, according to the present invention;

FIG. 2 is a perspective view of the infuser device according to the present invention seen from another angle;

FIG. 3 is a perspective view of the infuser device according to the present invention highlighting a main body decoupled from a removable group;

FIG. 4 is a side view of a detail of a peristaltic pump of the infuser device, according to the present invention;

FIG. 5 is a perspective view of a detail of a peristaltic pump of the infuser device, according to the present invention;

FIG. 6 is a graph of the trend of the weight of the liquid infused by a pump of the known type as a function of the rotation of the rotor;

FIG. 7 is a graph of the trend of the weight of the liquid infused by the pump according to the invention as a function of the rotation of the rotor.

### DETAILED DESCRIPTION OF THE INVENTION

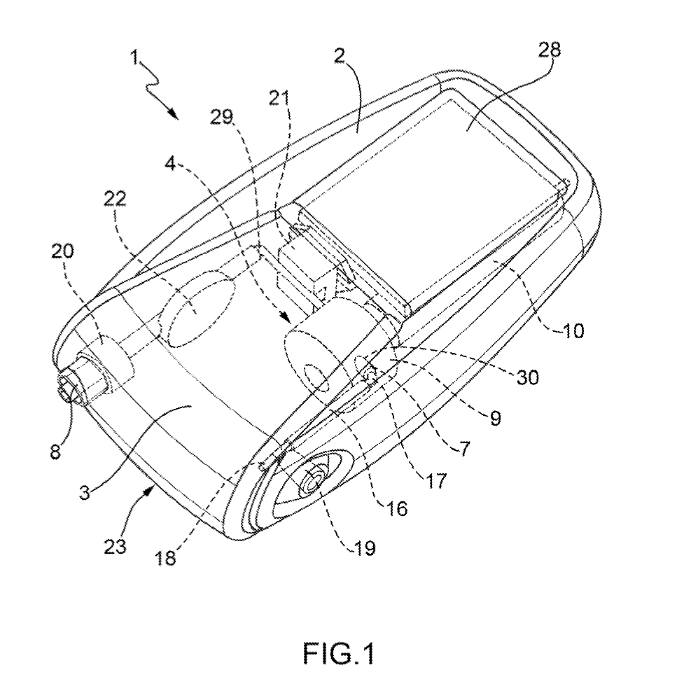

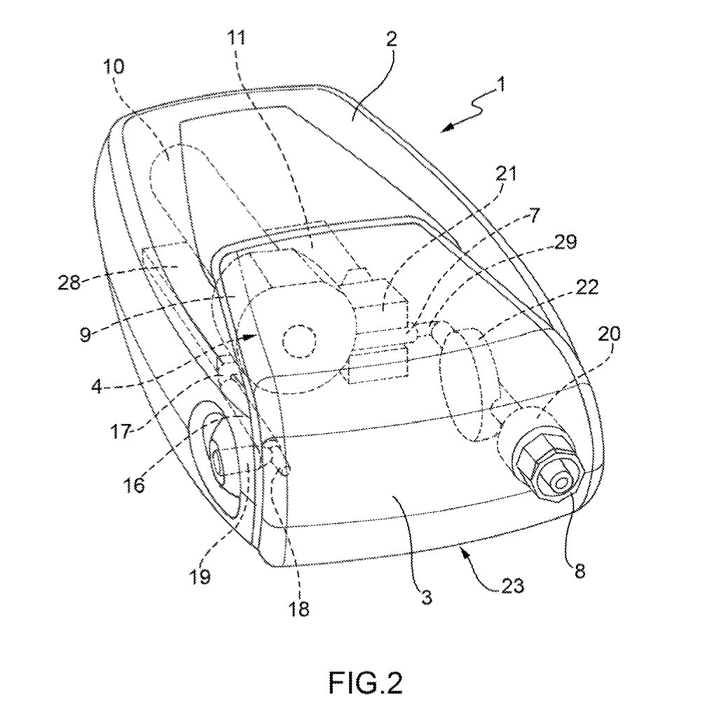

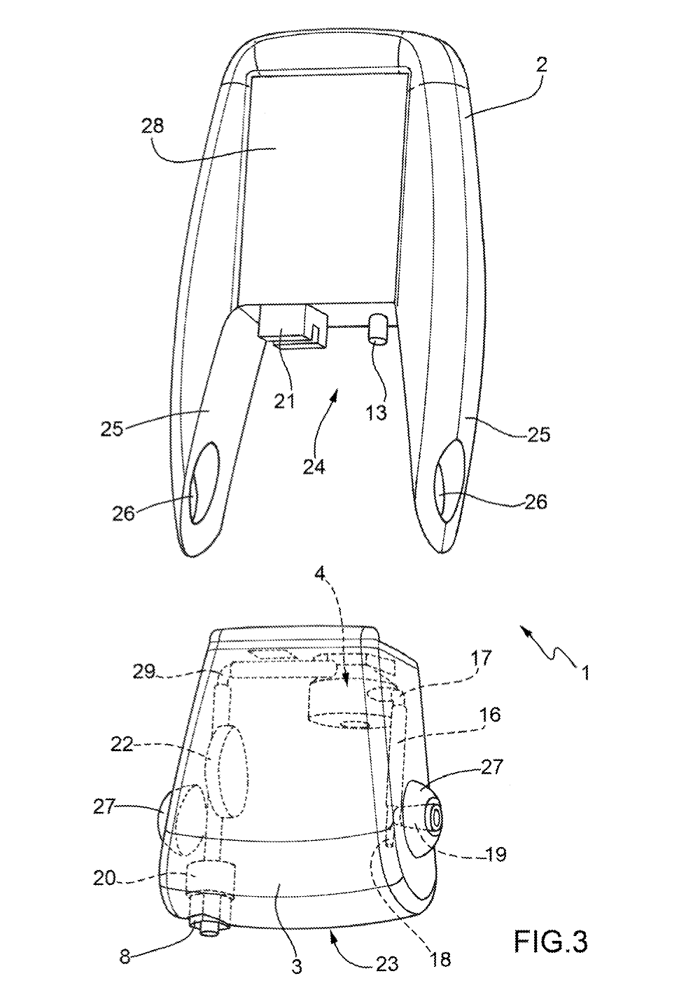

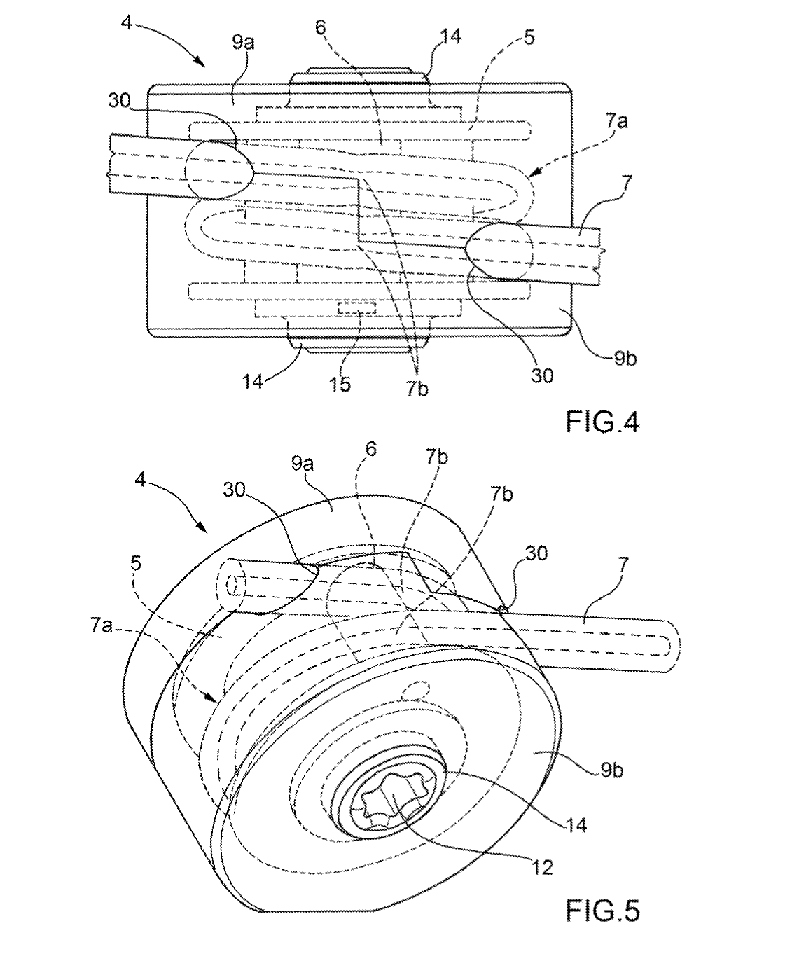

With reference to the attached FIG. 1, an infuser device for liquid medicines and the like according to the present invention is wholly indicated with reference numeral 1.

The infuser device 1 for liquid medicines and the like is of the type comprising at least one main body that is associated with at least one tank 3 of liquid medicine.

The infuser device 1 comprises at least one peristaltic pump 4 having at least one rotor 5 that is coupled with at least one press roller 6; the peristaltic pump 4 is associated with at least one tubular duct 7, made from elastically deformable material, placed in communication on one side with the tank 3, and on the other with at least one mouth 8 for dispensing the liquid of the infuser device 1.

The mentioned tubular duct 7 is wound so as to form at least one complete ring 7a abutting on the inner wall of at least one casing 9 of the peristaltic pump 4.

The roller 6 can be selectively pushed to engage with at least the complete ring 7a, so as to allow a continuous dispensing of liquid medicine for any flow-rate required.

More in detail, the single press roller 6 coupled with the rotor 5 is suitable for selectively pressing on the complete ring 7a of the tubular duct 7.

The press roller 6 can be coupled with the rotor 5 in a rotatable manner so as to not wear the tubular duct 7, i.e. in other words the press roller 6 can rotate around itself.

The rotor 5 is associated with at least one motor 10 that is slaved to at least one control processor 11 of the infuser device 1.

The control processor 11 comprises means for adjusting the speed of the rotor 5.

More in detail, such means for adjusting the speed make it possible to vary the speed of the rotor 5 during the same revolution, in other words in at least one sector of the complete turn carried out by the rotor 5.

Thanks to the presence of such means for adjusting the speed, as shall be described more clearly in the rest of the description, it is possible to carry out the adjustment of the rotation speed of the roller 6 and the consequent controlling of the dispensing over time of the liquid medicine at the required flow-rate.

More in detail, the dispensing of liquids can occur continuously, in a pulsed manner and in a mixed, pulsed and continuous manner, with a constant or variable operation.

Moreover, the rotor 5 comprises a shaped hole 12 for coupling with a respective shaft 13 of the motor 10.

The rotor 5 is housed rotatable inside the casing 9 of the peristaltic pump 4 on bushings 14 of the self-lubricating type.

The complete ring 7a comprises two portions that are substantially adjacent 7b of the tubular duct 7, at which a proximity sensor 15 is located that is associated with the control processor 11 and that is suitable for detecting the passage of the press roller 6.

For example the proximity sensor 15 can be of the optical or magnetic type.

The proximity sensor 15 can be positioned anywhere along the development of the tubular duct 7, as long as it is always kept in the same position.

In some simplified embodiments of the infuser device 1 according to the invention, the proximity sensor 15 may not be present, so as to achieve the control of the rotation of the rotor 5 in an open loop.

The tubular duct 7 is placed in communication with the tank 3 through a perforated rod 16 to which it is associated with a first angular joint 17; the perforated rod 16 comprises one suction end 18 that is anchored to a respective support element 19.

The infuser device 1 comprises a nonreturn valve 20 of the liquid medicine located downstream of the peristaltic pump 4, so as to prevent the return of liquid in the direction opposite to the dispensing direction.

Moreover the infuser device 1 may comprise a pressure sensor that is operatively connected to the control processor 11 and that is suitable for detecting the pressure of the liquid medicine.

The infuser device 1 can also comprise an air bubble detector.

Or, as illustrated in the figures, the infuser device 1 can comprise a single unit 21 for controlling the pressure of liquid medicine and the presence of air bubbles associated with the control processor 11.

It is then foreseen for there to be a filter 22 for expelling the air bubbles possibly present, so that they are not infused together with the liquid medicine dispensed to the patient.

According to an aspect of the present invention, the infuser device 1 comprises the mentioned main body 2 and moreover comprises a removable group 23 that can be coupled together.

More in detail the main body 2 houses the electronic components, and therefore at least the control processor 11, and at least the motor 10 for actuating the peristaltic pump 4, and at least one battery for supplying said electronic components; the removable group 23 on the other hand comprises at least the tank 3 and the peristaltic pump 4.

The main body 2 of the infuser device 1 defines a respective seat 24 for the removable group 23.

The seat 24 is suitable for allowing the corresponding coupling of the shaft 13 of the motor 10 with the rotor 5 of the peristaltic pump 4.

Moreover, the main body 2 comprises a pair of opposite appendages 25 defining shaped holes 26 for coupling respective projections 27 of the removable group 23.

At one of the projections 27, the already mentioned supporting element 19 for the perforated rod 16 is made inside the tank 3.

The main body 2 of the infuser device 1 supports also the control unit 21 that, in the coupled configuration of the main body 2 itself with the removable group 23, is associated with a portion of the tubular duct 7 so as to control the pressure of the liquid medicine and the possible presence of air bubbles.

The main body 2 of the infuser device 1 moreover houses a display 28 that is associated with the control processor 11, for the visualisation of information by the user. The control processor 11 can, for example, be made up of an electronic card housed in the main body 2.

The control processor 11 is suitable for communicating with external programming units through cabled, wireless or another kind of interfaces.

According to another aspect of the present invention, the removable group 23 is of the disposable type for obvious reasons of hygiene and safety for the patient.

As illustrated in the figures, the removable group 23 also houses the filter 22 for expelling the air bubbles, located downstream of the peristaltic pump 4 and associated with the tubular duct 7 through a second joint 29.

The tubular duct 7 is suitably made from an elastically deformable material, like for example silicone, PVC, or other polymeric material having suitable mechanical characteristics, which allows the quick return to the initial dimensions of the portions of tubular duct 7 pressed by the passing of the roller 6.

As mentioned the tubular duct 7 is wound in the complete ring 7a inside the casing 9, which is made up of two half-shells 9a, 9b defining, in the coupling configuration, two openings 30 for the passage of the tubular duct 7 itself.

In the following part, it is described the method for controlling the dispensing of liquid medicine and the like carried out with the infuser device 1 according to the present invention.

The method comprises a step of dividing the complete revolution carried out by the rotor 5 into a predetermined number of angular sectors.

The method then foresees a step of increasing, through the control processor 11, the rotation speed of the rotor 5 at least in the sector after the point in which the roller 6 passes at the adjacent portions 7b, defining, by the roller 6 itself, a faster compression of the corresponding ring portion 7a, so as to allow the continuous dispensing over time of the liquid medicine at the desired flow-rate.

The step of increasing the rotation speed of the rotor 5 can be actuated according to different modalities.

For example, the step can be actuated by providing, for example, an encoder or the like on the motor 10, and thus knowing the exact angular position of the rotor 5 at the passing of the roller 6 at the adjacent portions 7b of the tubular duct 7.

Or yet again, the detection of the passing of the press roller 6 at the adjacent portions 7b can be carried out with the measuring, again through the control processor 11, of the value of the current absorbed by the motor 10 during the first revolution of the rotor 5: the value of the absorbed current (which is proportional to the required torque) is substantially the maximum at the compression of the adjacent portions 7b by the roller 6 and it is substantially minimum in the portion of the tubular duct 7 that is immediately after the adjacent portions 7b, in such a way that the rotation speed of the rotor 5 is increased in the sector corresponding to the point of detection of the value of minimum absorbed current (i.e. rather immediately after the passing of the roller 6 at the adjacent portions 7b).

In the case in which the infuser device 1 is provided with the proximity sensor 15, the method according to the invention also comprises a step of detecting the passing of the press roller 6 at the adjacent positions 7b of the tubular duct 7.

The detection of the passing of the press roller 6 at the adjacent portions 7b is in this case carried out indeed through the proximity sensor 15, which is operatively connected to the control processor 11.

The method then foresees, as mentioned, a step of increasing, through the control processor 11, the rotation speed of the rotor 5 at least in the sector following the detection point, carried out by the sensor 15, of the passing of the roller 6 at the adjacent portions 7b, defining, by the roller 6 itself, a faster compression of the corresponding portion of the ring 7a, so as to allow the continuous dispensing over time of liquid medicine at the desired flow-rate.

The number of angular sectors in which each revolution of the rotor 5 is divided is for example equal to eight.

In such a way, the rotation speed of the rotor 5 and of the roller 6 in the sector following the passing point—possibly detected by the sensor 15—of the roller 6 itself at the adjacent portions 7b will be set higher through the control processor 11 with respect to the other seven sectors, leading to a faster compression by the roller 6 of the corresponding ring portion 7, so as to allow the continuous dispensing over time of the liquid medicine at the desired flow-rate.

The operation of the infuser device 1 for liquid medicine and the like, according to the invention is, according to what has been described, completely intuitive.

The liquid medicine contained in the tank 3 is sucked in the tubular duct 7, through the perforated rod 16, by the peristaltic pump 4 the rotor 5 of which, actuated by the motor 10, sets in rotation the roller 6 that selectively presses subsequent portions of the complete ring 7a against the cylindrical internal wall of the casing 9.

The selective action of the press roller 6 on the ring 7a made from elastically deformable material determines the forward movement due to peristaltic effect of the liquid medicine in outlet from the pump 4 towards the dispensing mouth 8 of the device; the ring portion 7a that has undergone compression by part of the roller 6 immediately after returns to the initial sizes leading to drawing of liquid from the tank.

As mentioned the control processor 11 controls the rotation speed of the motor 10, and therefore of the press roller 6, for predetermined values along the mentioned eight angular sectors so as to continuously dispense over time the liquid medicine, according to the required flow-rate.

In particular the rotation speed is as mentioned faster in the sector following the point in which the rollers 6 passes at the mentioned adjacent portions 7b of the ring 7a. Indeed, if the speed of the rotor were always the same for the entire revolution, when the roller 6 passes just beyond the adjacent portions 7b, the dispensing of liquid would stop briefly due to the fact that space is freed up on the delivery side which is immediately filled by the liquid moving forward (the nonreturn valve 20 prevents as mentioned the return of liquid already pushed out by the pump 4 in the direction opposite to the dispensing direction): therefore, even if the total amount of dispensed liquid for each complete revolution of the rotor 5 in any case remains the same, there would be a slowing down of the dispensed flow i.e. a non continuous dispensing over time of the liquid medicine.

In order to avoid such a drawback and to allow the continuous dispensing over time of the liquid medicine, it is necessary to act, as seen, upon the speed of the rotor 5, and therefore of the roller 6, which is maximum in the sector following the detection point of the passing of the roller 6 at the adjacent portions 7b.

Such a passing of the roller 6 is detected as mentioned through the proximity sensor 15 or by measuring the values of the current absorbed by the motor 10 during the first complete revolution of the rotor 5, or with yet other methods.

It is obvious how by controlling the rotation speed of the rotor 5 through the processor 11 it is possible to obtain the continuous dispensing of the liquid at any flow-rate required.

The invention, thus conceived, makes it possible to obtain important technical advantages.

An important technical advantage consists of the fact that the infuser device 1 allows a dispensing in outlet of the liquid medicine that is continuous and having any flow-rate required, for the optimal dosage of the infused liquid, the precision of which is an essential requirement in the medical field.

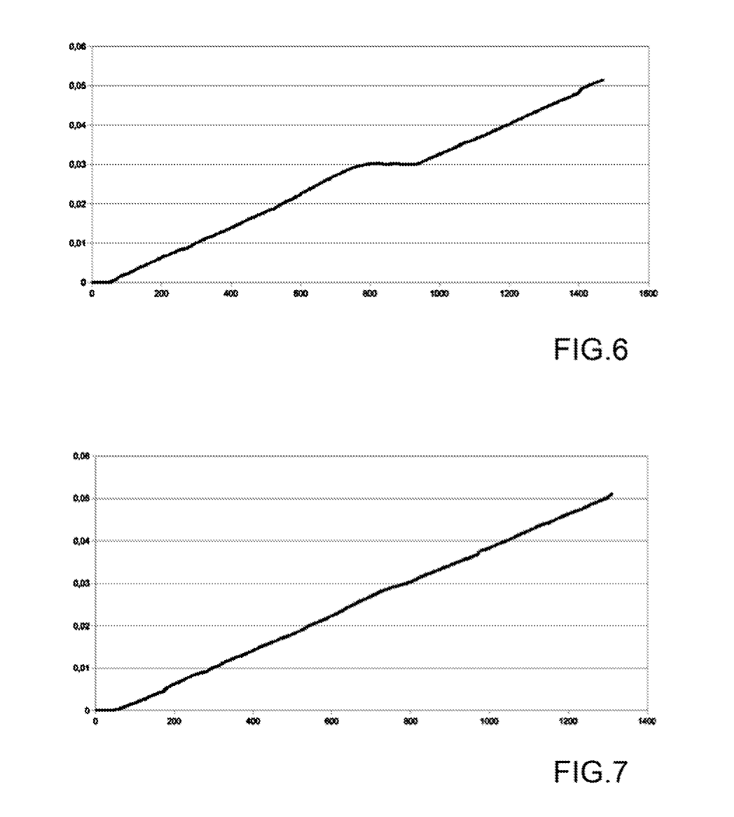

Attached FIGS. 6 and 7 respectively show two graphs in which in the y-axis there is the weight of the dispensed liquid in grams, whereas in the x-axis there is the time needed for a single rotation.

More in detail, the graph of FIG. 6 shows the rotation/weight trend relative to a peristaltic pump without means for adjusting the speed, whereas figure shows the rotation/weight trend relative to a peristaltic pump with means for adjusting the speed according to the present invention.

As can be observed, the speed adjustment means make it possible to restore the linearity in the dispensing of the peristaltic pump 4.

The effect of the speed adjustment means, and therefore of the increase of the speed at a sector of the rotation of the peristaltic pump 4, is greater when the required dosage is lower.

Moreover, the infuser device 1 comprises a single roller 6, with advantages also in terms of cost and in manufacturing simplicity.

Moreover, the device comprises a disposable removable group 23 comprising the peristaltic pump 4 and the tank 3, such a characteristic ensuring maximum safety in terms of hygiene, against possible risk of contamination.

It is moreover worth underlining that the division in angular sectors of the complete revolution carried out by the rotor 5 makes it possible to avoid the situations in which, due to possible problems of electronic control or of programming, the pump operates in such a manner as to not respect the commands of the control system.

In this particular case, each movement command in every sector in which the complete revolution is divided is only generated when the previous command has been completed.

In one embodiment of the infuser device according to the present invention, voice message generation means are foreseen, which can be heard by the user, which inform the patient and operators, in real time, on the various operation conditions of the peristaltic pump, on the setting of the parameters, on possible faults, etcetera.

The means for generating voice messages are operatively connected to the control processor 11 of the device.

One example in which the means for generating voice messages can be applied is, for example, a situation in which it is necessary to reprogram the pump for a different therapy. In this case, the operator types on the display 28, for example, the base infusion values, bolus values, and the lock-out time, or rather the set time interval between two incremental doses.

Once the programming has finished, the operator can require the voice confirmation: the voice repeats the values inserted, highlighting them on the display 28 at the same time.

The operator can in this way be sure that the parameters he meant to insert have actually been inserted and are correct.

Another example of application can be the possibility of requiring a voice confirmation on the correct operation of the pump, on the autonomy of the batteries, on the residual duration of the therapy, and the like, by pressing a suitable button, or icon, on the display 28.

Other examples of application can be the generation of voice messages relative to situations of emergency due to faults or other situations like low autonomy of the batteries, depletion of the drug, possible obstruction of the infusion line, and the like.

In another embodiment of the present invention, the infuser device comprises means for automatically correcting the flow-rate dispensed by the peristaltic pump 4 in relation to the size errors found on the inner diameter of the disposable tubular duct 7.

The correction means comprise a feeler, of the per se known type, the operation of which shall become clearer in the rest of the description.

Indeed, it has experimentally been found that the tolerance on the inner diameter of the tubular duct 7 has a great influence on the dispensing precision of the peristaltic pump 4.

Only as an example it should be considered that, if the inner diameter of the tubular duct 7 is of 1.05 mm instead of 1 mm, a consequent variation of about 9.4% of the section of the tube leads to a variation of 7.5% of the quantity of drug dispensed.

This difference with respect to the nominal parameters is too high.

In addition to pre-emptively eliminating the tubes with sizes out of tolerance, the infuser device according to the invention, through the aforementioned correction means, makes it possible to automatically adjust the flow-rate dispensed by the peristaltic pump 4 compensating the size error of the actual inner diameter of the tubular duct 7 with respect to the nominal value of the aforementioned inner diameter.

In practice, during production of the tubular duct 7, its inner diameter is verified and a suitable label is placed on the duct indicating the correction factor that the operator must insert into the management interface of the operation of the infuser device—i.e. to operate with that specific tubular duct 7.

Moreover, in the same disposable tubular duct 7, again during production, a blind hole is made having a depth that is proportional to the difference in size measured in the inner diameter with respect to the nominal diameter.

When the disposable tubular duct 7 is inserted inside the infuser device according to the invention, the feeler measures the depth of the blind hole and automatically sets the correction factor for the operation of the peristaltic pump 4.

With this provision it is therefore possible—if we do not consider errors in measuring the inner diameter of the tube—to compensate, and thus eliminate, the error of dispensing of the peristaltic pump 4 due to the difference in sizes of the disposable tubular ducts 7.

It has thus been seen how the invention achieves the proposed purposes.

The present invention has been described according to preferred embodiments, but equivalent variants can be conceived without for this reason departing from the scope of protection offered by the following claims.

1. A universal infusion device for liquid medicines and the like, comprising at least a main body associated to at least a tank of liquid medicine, at least a peristaltic pump having at least a rotor coupled with at least a pressing roller pivotable on itself which can be engaged by pushing on a tubular duct made of elastically deformable material arranged in communication on one hand with said tank and on the other hand with at least a mouth for dispensing the liquid of said infusion device, said tubular duct being wrapped to make up at least a complete ring abutting on the inner wall of at least a shell of said peristaltic pump and comprising two portions substantially arranged one adjacent to the other of said tubular duct, said rotor being coupled with a motor operatively connected to a control processor, characterised in that said control processor comprises means for adjusting the rotational speed of said rotor adapted to increase the speed of said rotor in at least a sector of the complete revolution performed by said rotor.

2. The device according to claim 1, wherein said adjusting means are adapted to increase the rotational speed of said rotor in the section subsequent to the passage of said pressing roller at said portions arranged adjacent to one another, so as to allow the liquid medicine to be dispensed for any flow rate required.

3. The device according to claim 2, wherein the dispensing of the liquid by said peristaltic pump can occur in a continuous manner, in a pulsed manner or in a mixed manner—pulsed and continuous—, under constant or variable state.

4. The device, according to claim 1, wherein said rotor comprises at least a shaped hole for being coupled with at least a respective shaft of said motor.

5. The device, according to claim 1, comprising at least a proximity sensor located at said two portions arranged substantially adjacent to one another of said tubular duct, said proximity sensor being associated with said control processor and adapted to detect the passage of said pressing roller.

6. The device, according to claim 1, comprising a check valve of the liquid medicine located downstream of said peristaltic pump so as to prevent the liquid from returning in the direction opposite to that of dispensing.

7. The device, according to claim 1, comprising at least a pressure sensor associated with said control processor and adapted to detect the pressure of the liquid medicine.

8. The device, according to claim 1, further comprising at least a detector of air bubbles.

9. The device, according to claim 1, comprising a unit for monitoring the liquid medicine pressure and the presence of air bubbles associated with said control processor.

10. The device, according to claim 1, further comprising at least a filter for ejecting air bubbles.

11. The device, according to claim 1, comprising a removable group, comprising at least said tank and at least said peristaltic pump, which can be coupled to said main body, said main body housing at least said motor and said control processor.

12. The device, according to claim 11, wherein said main body defines at least a seat for said removable group adapted to allow the corresponding coupling of said shaft of said motor of said peristaltic pump, said main body comprising a pair of opposite extensions defining shaped holes for coupling to respective projections of said removable group.

13. The device, according to claim 11, wherein said removable group is of the disposable type.

14. The device, according to claim 1, comprising at least a display housed in said main body and associated with said control processor at least for displaying information.

15. The device, according to claim 1, wherein said control processor is an electronic card housed in said main body.

16. The device, according to claim 7, wherein said tubular duct is made of an elastically deformable material selected from among silicone, PVC, and other polymeric materials.

17. The device, according to claim 1, wherein said shell consists of two half-shells defining in a coupling configuration at least two openings for the passage of said tubular duct.

18. The device according to claim 1, comprising means for generating voice messages, relating to the operating state of the device, operatively connected to said control processor.

19. The device according to claim 1, wherein said control processor is adapted to dialogue with external programming units through wired or wireless interfaces.

20. The according to claim 1, further comprising means for automatically adjusting the flow rate dispensed by said peristaltic pump relating to the size errors detected on the inner diameter of said disposable tubular duct.

21. The according to claim 20, wherein said automatic adjusting means comprise a feeler adapted to be introduced in a blind hole drilled in said disposable tubular duct having a depth proportional to the size gap between the actual inner diameter and the nominal inner diameter.

22. A method for controlling the dispensing of liquid medicine, comprising the steps of:
dividing the complete revolution performed by a rotor in a predetermined number of angular sectors;
increasing through a control processor the rotational speed of said rotor at least in a sector subsequent to the passage point of a roller at portions arranged adjacent to one another, resulting in a faster compression by said roller of the corresponding portion of a ring so as to allow for the continuous dispensing over time of the liquid medicine at a desired flow rate.

23. The method according to claim 22, further comprising a step of detecting the passage of said roller at said portions arranged adjacent to one another of a tubular duct.

24. The method according to claim 22, wherein said step of detecting the passage of said roller at said portions arranged adjacent to one another is carried out through a proximity sensor associated with a control processor.

25. The method according to claim 22, wherein said step of detecting the passage of said roller at said portions arranged adjacent to one another is carried out by measuring, through at least a control processor the value of the current absorbed by said motor during the first revolution of said rotor, the value of the absorbed current being substantially maximum upon compressing said portions arranged adjacent to one another by said roller and being substantially minimum in the section of said tubular duct subsequent to said portions arranged adjacent to one another, so as to increase the rotational speed of said rotor at least in the sector corresponding to the point of detecting the minimum value of the absorbed current.

26. The method according to claim 22, wherein the number of said angular sectors is eight.
